(19) United States
(12) Patent Application Publication
(10) Pub. No.: US 2025/0002512 A1
(43) Pub. Date: Jan. 2, 2025

(54) NOVEL ANTI-DEPRESSANT AND NEUROPLASTIC AGENTS AND THERAPEUTIC USES THEREOF

(71) Applicant: MARVEL BIOTECHNOLOGY, Calgary (CA)

(72) Inventor: Mark WILLIAMS, Calgary (CA)

(73) Assignee: MARVEL BIOTECHNOLOGY, Calgary, AB (CA)

(21) Appl. No.: 18/703,742

(22) PCT Filed: Oct. 26, 2022
(86) PCT No.: PCT/CA2022/051579

§ 371 Date: Apr. 23, 2024

Related U.S. Application Data
(60) Provisional application No. 63/272,462, filed on Oct. 27, 2021.

(51) Int. Cl. C07F 9/6558; A61K 31/4045; A61K 31/675; A61P 25/24; C07D 403/12

(52) U.S. Cl. CPC C07F 9/65583; A61K 31/4045; A61K 31/675; A61P 25/24; C07D 403/12

(57) ABSTRACT

A compound of Formula (6): Formula (8): and Formula (10): are prepared and found useful in the treatment and/or prevention of diseases and conditions such as Parkinson's disease, amyotrophic lateral sclerosis, Alzheimer's disease, fetal alcohol exposure, autism, schizophrenia, traumatic brain injury, stroke, addictions, Huntington's disease, Fragile X and Rett's syndrome.

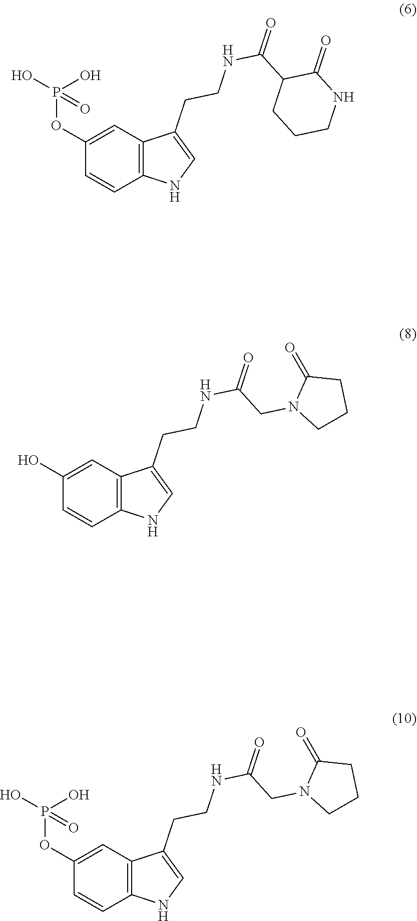

### CROSS REFERENCE TO RELATED APPLICATIONS

The present application claims priority from U.S. provisional application No. 63/272,462, filed Oct. 27, 2021.

### FIELD OF THE INVENTION

The present invention is related to compounds having anti-depressant and neuroplastic properties, pharmaceutical compositions comprising these compounds, and their use in the treatment of depression diseases associated with neuronal damage, in particular for humans.

### BACKGROUND OF THE INVENTION

Depression is a significant medical problem despite a large number of medications already approved for its treatment. As many as a third of patients do not respond to current anti-depressants, and if they do respond, beneficial effects from treatment can take at least 2-4 weeks before improvement is felt. Interest has grown in the psychedelic space because of the ability of these molecules such as ketamine, LSD, NMDA and tryptamines (DMT, psilocybin) because of their ability to promote neuroplasticity and rapidly reduce depressive symptoms (Ly C, Greb A C, Cameron L P, et al. Psychedelics Promote Structural and Functional Neural Plasticity. Cell Rep. 2018; 23 (11): 3170-3182. doi: 10.1016/j.celrep.2018.05.022). There are several mechanisms, but brain derived neurotrophic factor (BDNF) and its receptor TrkB are intimately involved.

The ability to promote neuroplasticity is potentially important to neurological diseases beyond depression and its related conditions alone and could positively and broadly effect other diseases including but not limited to: Parkinson's disease, amyotrophic lateral sclerosis, Alzheimer's disease, fetal alcohol exposure, autism, schizophrenia (C L, CB C, K Y. 7,8-dihydroxyflavone, a small molecular TrkB agonist, is useful for treating various BDNF-implicated human disorders. Transl Neurodegener. 2016; 5 (1). doi: 10.1186/S40035-015-0048-7). In view of the clinical importance of promoting neuroplasticity, there is a need for the identification of compounds that promote neuroplasticity for the development of new therapeutic agents. Such compounds are provided herein.

### SUMMARY OF THE INVENTION

One aspect of this invention concerns the compound of Formula (6):

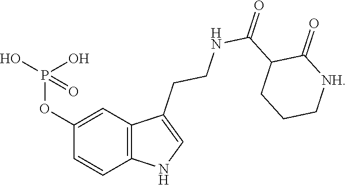 [SMILES: O=C1NCCCC1C(=O)NCCc1c[nH]c2ccc(OP(=O)(O)O)cc12]

Another aspect of this invention concerns the compound having the Formula (8):

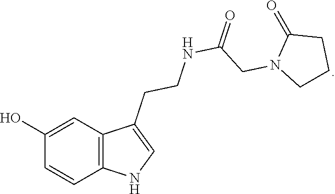 [SMILES: O=C(CN1CCCC1=O)NCCc1c[nH]c2ccc(O)cc12]

Another aspect of this invention concerns the compound having the Formula (10):

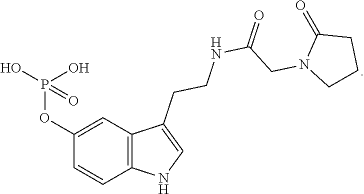 [SMILES: O=C(CN1CCCC1=O)NCCc1c[nH]c2ccc(OP(=O)(O)O)cc12]

The invention further teaches the use of the compounds of Formula (6) (8) and (10) for the treatment of depression. The invention further teaches the use of the compounds of Formula (6) (8) and (10) for the treatment of one of more of Parkinson's disease, amyotrophic lateral sclerosis, Alzheimer's disease, fetal alcohol exposure, autism, schizophrenia, traumatic brain injury, stroke, addictions, Huntington's disease, Fragile X and Rett's syndrome.

### BRIEF DESCRIPTION OF THE DRAWINGS

The present invention will now be described in more detail having regard to the drawings in which:

FIG. 1 shows a chart representation of immobility time of test compounds, vehicle control, and a positive control antagonist.

FIG. 2 shows a chart representation of time/latency to immobility of test compounds, vehicle control, and a positive control antagonist.

### DETAILED DESCRIPTION

The invention may be more fully appreciated by reference to the following description, including the following glossary of terms and the concluding examples.

Throughout the following description, specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

The present inventors have synthesized and tested novel molecules possessing the tryptamine pharmacophore in an animal model of depression as prototypic example of their use. This is an important finding for treatment and/or prevention of these conditions.

In preferred embodiments, the disease, disorder, or medical condition is selected from: neurologic and psychiatric disorders including, but not limited to: mood disorders and mood affective disorders, depression, Parkinson's disease, amyotrophic lateral sclerosis, Alzheimer's disease, fetal alcohol exposure, autism, traumatic brain injury, stroke, addictions, Huntington's disease, Fragile X and Rett's syndrome and schizophrenia, acute and chronic; and any sequelae of cerebrovascular diseases.

The term “treat”, “treatment” or “treating”, as used herein, is intended to refer to administration of an active agent or composition according to an embodiment of the invention to a subject for the purpose of affecting a therapeutic or prophylactic benefit through modulation of neuroplasticity. Treating includes reversing, ameliorating, alleviating, inhibiting the progress of, lessening the severity of, or preventing a disease, disorder, or condition, or one or more symptoms of such disease, disorder or condition mediated through modulation of neuroplastic activity. The term “subject” refers to a mammalian patient in need of such treatment, such as a human.

The term “modulating” encompasses increasing, enhancing, inhibiting, decreasing, suppressing, and the like, generally in a physiologically significant manner.

The expression “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

The term “effective amount” refers to that amount of an active agent or compound that is being administered or that is to be administered, which is sufficient to prevent the disease, disorder, or medical condition, or prevent one or more symptoms of the disease, disorder, or medical condition being treated. In certain embodiments, the term “effective amount” refers to that amount of an active agent or compound that is being administered or that is to be administered, which is sufficient to reduce the risk of the disease, disorder, or medical condition, or one or more symptoms of the disease, disorder, or medical condition. Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day, or about 0.2 to about 2.5 g/day.

In addition, the compounds of the invention may be used in combination with additional active ingredients in the treatment of the above conditions. The additional active ingredients may be co-administered separately for concomitant or sequential administration, or included with such an agent in a pharmaceutical composition according to the invention. In an exemplary embodiment, additional active ingredients are those that are known or discovered to be effective in the treatment of conditions, disorders, or diseases mediated by neuroplastic activity, including those noted above, such as another neuroplastic modulator or a compound active against another target associated with the particular condition, disorder, or disease. The combination may serve to increase efficacy (e.g., by including in the combination a compound potentiating the potency or effectiveness of an active agent according to the invention), decrease one or more side effects, or decrease the required dose of the active agent according to the invention.

The compounds of the invention may be used, alone or in combination with one or more additional active ingredients, to formulate pharmaceutical compositions of the invention. A pharmaceutical composition of the invention comprises: (a) an effective amount of at least one compound in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Also included are disintegrating agents, binding agents, lubricating agents, sweetening agents, flavoring agents, coloring agents and preservative agents. Examples of excipients sodium and calcium carbonate, sodium and calcium phosphate, lactose, starch, sugar, glucose, cellulose derivatives including methyl cellulose, magnesium stearate, polyethylene glycol, mannitol, sorbitol, and the like. Exemplary liquid oral excipients include ethanol, glycerol, water, vegetable oils, and the like. Starch, polyvinyl-pyrrolidone (PVP), sodium starch glycolate, microcrystalline cellulose, and alginic acid are suitable disintegrating agents. Binding agents may include starch and gelatin. The lubricating agent, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Delivery forms of the pharmaceutical compositions containing one or more dosage units of a compound according to the present invention as an active agent may be prepared using suitable pharmaceutical excipients and compounding techniques known or that become available to those skilled in the art. The compositions may be administered in the by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the compounds may be formulated to yield a dosage of, e.g., from about 0.05 to about 100 mg/kg daily, or from about 0.05 to about 35 mg/kg daily, or from about 0.1 to about 10 mg/kg daily. For example, a total daily dosage of about 5 mg to 5 g daily may be accomplished by dosing once, twice, three, or four times per day.

Capsules for oral administration include hard and soft gelatin capsules. To prepare hard gelatin capsules, compounds of the invention may be mixed with a solid, semi-solid, or liquid diluent. Soft gelatin capsules may be prepared by mixing the compound of the invention with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compounds of this invention may also be administered by non-oral routes. For example, the compositions may be formulated for rectal administration as a suppository. For parenteral use, including intravenous, intramuscular, intraperitoneal, or subcutaneous routes, the compounds of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms are presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses may range from about 1 to 1000 mcg/kg/minute of compound, admixed with a pharmaceutical carrier over a period ranging from several minutes to several days.

For topical administration, the compounds may be mixed with a pharmaceutical carrier at a concentration of about 0.1% to about 10% of drug to vehicle. Another mode of administering the compounds of the invention may utilize a patch formulation to affect transdermal delivery. Compounds of the invention may alternatively be administered in methods of this invention by inhalation, via the nasal or oral routes, e.g., in a spray formulation also containing a suitable carrier.

### Examples

### Example 1-Synthesis of N-[2-(5-hydroxy-1H-indol-3-yl)ethyl]-2-oxo-3-piperidinecarboxamide (1)

This compound is a phosphorylated derivative of the compound N-[2-(5-hydroxy-1H-indol-3-yl)ethyl]-2-oxopiperidine-3-carboxyamide (aka HIOC, CAS #314054-36-9) which has been shown to activate the brain derived neurotrophic factor (BDNF) receptor TrkB, a pathway known to promote neuroplasticityl and neuroprotection (Shen J, Ghai K, Sompol P, et al. N-acetyl serotonin derivatives as potent neuroprotectants for retinas. doi: 10.1073/pnas.1119201109). The compound is normally given via intraperitoneal injection as the compound bears a catechol structure which is highly susceptible to first-pass metabolism such as O-glucuronidation (reference). By phosphorylating the catechol, the compound is orally active and water soluble, both desired qualities in a drug.

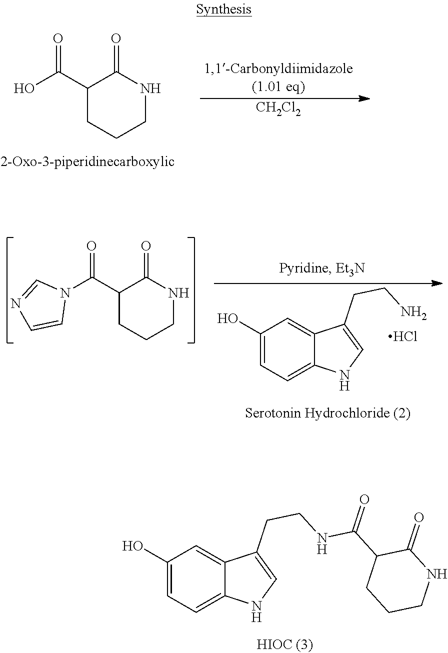

An oven-dried round-bottomed flask was equipped with a stir bar and charged with 2-oxopiperidine-3-carboxylic acid (1, 0.565 g, 3.95 mmol, 1.00 eq) and CH₂Cl₂ (13 mL). The mixture was purged with argon, and carbonyldiimidazole (0.647 g, 3.99 mmol, 1.01 eq) was added in one portion. The resulting mixture was stirred at room temperature for 30 min, where upon pyridine (13 mL) was added, followed by serotonin-HCl (2, 0.857 g, 4.03 mmol, 1.02 eq). Upon complete dissolution of serotonin (˜10-15 minutes), triethylamine (1.0 mL, 7.90 mmol, 2.00 eq) was added. The reaction mixture was stirred at room temperature for another 3 h and concentrated in vacuo, with heating to 40° C. and azeotroping with several portions of toluene (˜50 mL in total) to ensure complete removal of the pyridine. The resulting gum was purified using flash chromatography, and product was eluted with 90:10 EtOAc/MeOH. The relevant fractions (Rf 0.22 in 90:10 EtOAc/MeOH) were combined and concentrated in vacuo. The resulting solid was loaded onto a glass frit and washed with 150 ml of warm diethyl ether, in order to remove any traces of imidazole co-eluted in column chromatography, to furnish N-[2-(5-Hydroxy-1H-indol-3-yl)ethyl]-2-oxo-3-piperidinecarboxamide (HIOC, 3) as a light violet solid (1.04 g, 87%). MS (ES+ve) Calcd for C₁₆H₂₀N₃O₃ [M+H]⁺: 302.34; Found: 302.41.

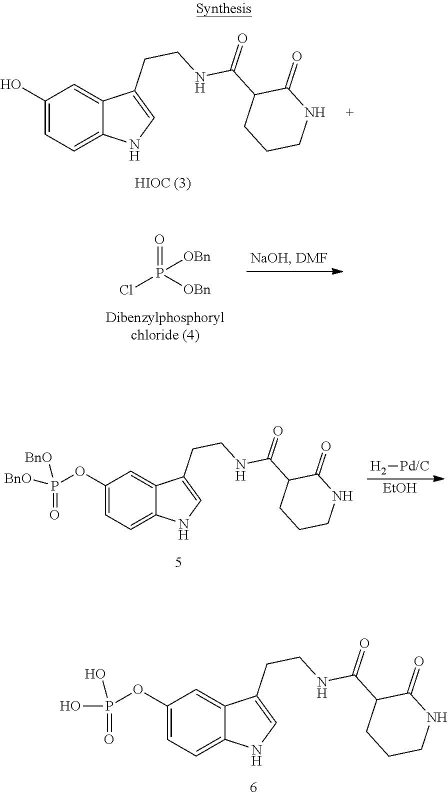

An oven-dried round-bottomed flask was equipped with a stir bar was charged with HIOC (3, 0.301 g, 1.00 mmol, 1.0 eq) and DMF (5 mL). The mixture was purged with argon, and NaOH (0.06 g, 1.5 mmol, 1.5 eq) previously dissolved in minimum amount of water (˜0.1 mL) was added dropwise. The mixture was stirred at room temperature for 15 minutes. Subsequently, freshly prepared dibenzylphosphoryl chloride (4, 347 μL, 1.5 mmol, 1.5 eq) was added dropwise under argon atmosphere at 0° C. The reaction mixture was warmed to room temperature and stirred for additional 1 h and concentrated in vacuo, with heating at 45° C. to ensure complete removal of the DMF. The resulting gum was purified using flash chromatography, and product was eluted with CH₂Cl₂/MeOH (92:8). The relevant fractions (Rf 0.45 in CH₂Cl₂/MeOH; 90:10) were combined and concentrated in vacuo to furnish N-{2-[5-(dibenzyloxyphosphoryloxy)-1H-indol-3-yl]ethyl}-2-oxo-3-piperidinecarboxamide (5) as a colorless oil (323 mg, 58%). MS (ES+ve) Calcd for C₃₀H₃₃N₃O₆P [M+H]⁺: 562.56; Found: 562.50.

To a stirred solution of the N-{2-[5-(dibenzyloxyphosphoryloxy)-1H-indol-3-yl]ethyl}-2-oxo-3-piperidinecarboxamide (5, 200 mg, 0.56 mmol) in 10 mL of EtOH was added 10% Pd—C (114 mg) and stirred under hydrogen atmosphere (balloon) at room temperature for 3 h hours. The reaction mixture was then filtered through celite and concentrated in vacuo. Trituration from ethyl acetate/hexane afforded the light brown solid which was dried under high vacuum to get the N-{2-[5-(Dihydroxyphosphoryloxy)-1H-indol-3-yl]ethyl}-2-oxo-3-piperidinecarboxamide (6, 118 mg, 87%). MS (ES+ve) Calcd for C₁₆H₁₉N₃O₆P [M−H]⁺: 380.32; Found: 380.60. ¹H-NMR (CD3OD; 400 MHZ): δ (ppm) 7.40 (s, 1H), 7.26 (d, J=11.2 Hz, 1H), 7.13 (s, 1H), 6.99 (d, J=11.2 Hz, 1H), 3.50 (t, J=9.0 Hz, 2H), 3.33-3.29 (m, 1H), 3.22 (t, J=6.8 Hz, 2H), 2.93 (t, J=8.8 Hz, 2H), 2.06-1.59 (m, 4H); ³¹P-NMR (CD₃OD; 121 MHZ): δ (ppm) −3.96.

### Example 2-Synthesis of N-{2-[5-(dihydroxyphosphoryloxy)-1H-indol-3-yl]ethyl}(2-oxo-1-pyrrolidinyl) acetamide (2)

Similar to HIOC, this novel compound is a phosphorylated N-acetyl serotonin derivative for neuroplasticity (Jang S W, Liu X, Pradoldej S, et al. N-acetylserotonin activates TrkB receptor in a circadian rhythm. Proc Natl Acad Sci USA. 2010; 107 (8): 3876-3881. doi: 10.1073/pnas.0912531107). It too has a catechol core structure which was protected with the phosphate group.

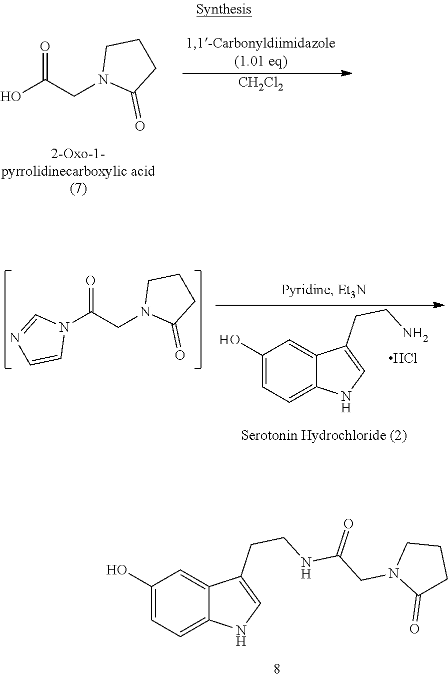

An oven-dried round-bottomed flask was equipped with a stir bar and charged with (2-Oxo-1-pyrrolidinyl) acetic acid (7, 0.401 g, 2.80 mmol, 1.00 eq) and CH₂Cl₂ (10 mL). The mixture was purged with argon, and carbonyldiimidazole (0.500 g, 3.08 mmol, 1.01 eq) was added in one portion. The resulting mixture was stirred at room temperature for 30 min, where upon pyridine (10 mL) was added, followed by serotonin-HCl (2, 0.607 g, 2.86 mmol, 1.02 eq). Upon complete dissolution of serotonin (˜10-15 minutes), triethylamine (0.7 mL, 5.60 mmol, 2.00 eq) was added. The reaction mixture was stirred at room temperature for another 3 h and concentrated in vacuo, with heating to 40° C. and azeotroping with several portions of toluene (˜45 mL in total) to ensure complete removal of the pyridine. The resulting gum was purified using flash chromatography, and product was eluted with 90:10 EtOAc/MeOH. The relevant fractions (Rf 0.22 in 90:10 EtOAc/MeOH) were combined and concentrated in vacuo. The resulting solid was loaded onto a glass frit and washed with 150 mL of warm diethyl ether, in order to remove any traces of imidazole co-eluted in column chromatography, to furnish N-[2-(5-hydroxy-1H-indol-3-yl) ethyl](2-oxo-1-pyrrolidinyl) acetamide (8) as a light violet solid slightly contaminated with imidazole. MS (ES+ve) Calcd for C₁₆H₂₀N₃O₃ [M+H]⁺: 302.34; Found: 302.30.

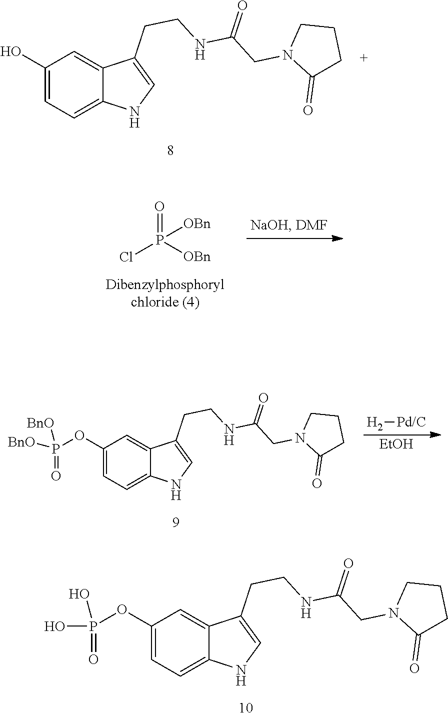

An oven-dried round-bottomed flask was equipped with a stir bar was charged with N-[2-(5-hydroxy-1H-indol-3-yl) ethyl] (2-oxo-1-pyrrolidinyl) acetamide (8, 1.00 g, 3.32 mmol, 1.0 eq) and DMF (10 mL). The mixture was purged with argon, and NaOH (0.200 g, 4.98 mmol, 1.5 eq) previously dissolved in minimum amount of water (˜0.2 mL) was added dropwise. The mixture was stirred at room temperature for 15 minutes. Subsequently, freshly prepared dibenzylphosphoryl chloride (4, 1.54 mL, 6.64 mmol, 1.5 eq) was added dropwise under argon atmosphere at 0° C. The reaction mixture was warmed to room temperature and stirred for additional 1 h and concentrated in vacuo, with heating at 45° C. to ensure complete removal of the DMF. The resulting gum was purified using flash chromatography, and product was eluted with EtOAc/MeOH (85:15). The relevant fractions were combined and concentrated in vacuo to furnish N-{2-[5-(dibenzyloxyphosphoryloxy)-1H-indol-3-yl]ethyl}(2-oxo-1-pyrrolidinyl) acetamide (9) as a colorless oil (705 mg, 44% over two steps). MS (ES+ve) Calcd for C₃₀H₃₃N₃O₆P [M+H]⁺: 562.56; Found: 562.40.

To a stirred solution of the N-{2-[5-(dibenzyloxyphosphoryloxy)-1H-indol-3-yl]ethyl}(2-oxo-1-pyrrolidinyl) acetamide (9, 397 mg, 0.71 mmol) in 15 mL of EtOH was added 10% Pd—C (225 mg) and stirred under hydrogen atmosphere (balloon) at room temperature for 3 h hours. The reaction mixture was then filtered through celite and concentrated in vacuo. Trituration from ethyl acetate/hexane afforded the light brown solid which was dried under high vacuum to get the N-{2-[5-(dihydroxyphosphoryloxy)-1H-indol-3-yl]ethyl} (2-oxo-1-pyrrolidinyl) acetamide (10, 208 mg, 78%). MS (ES+ve) Calcd for C₁₆H₁₉N₃O₆P [M−H]⁺: 380.32; Found: 380.60. ¹H-NMR (D20; 400 MHZ): δ (ppm) 7.44-7.41 (m, 2H), 7.23 (s, 1H), 7.05 (d, J=11.6 Hz, 1H), 3.83 (s, 2H), 3.54 (t, J=8.8 Hz, 2H), 3.10 (t, J=9.6 Hz, 2H), 2.97 (t, J=8.8 Hz, 2H), 2.36 (t, J=10.8 Hz, 2H), 1.91 (quintet, J=10.3 Hz, 2H); ³¹P-NMR (D20; 121 MHZ): δ (ppm) −3.50.

### Example 3—In Vivo Testing of the Compounds (1) and (2) in a Preclinical Model of Depression

The forced swim test (FST) is a widely accepted pre-clinical model to test the anti-depressive effects of molecules (Can A, Dao D T, Arad M, Terrillion C E, Piantadosi S C, Gould T D. The Mouse Forced Swim Test. J Vis Exp. 2012; (59): 3638. doi: 10.3791/3638).

Briefly, male CD-1 mice (approximately 25-35 g, n=8/group) were dosed orally once a day with either vehicle (2% aqueous DMSO, 0.5% carboxymethyl cellulose), compound 1 (50 mgs/kg in vehicle), compound 2 (50 mgs/kg in vehicle) or fluoxetine (aka. Prozac®, 30 mg/kg in vehicle) for seven days. Seven days is a comparatively short time to observe an anti-depressant effect with an orally dosed drug. On the seventh day, animals were tested in the FST and observed for abnormal behaviours (e.g. hallucinating).

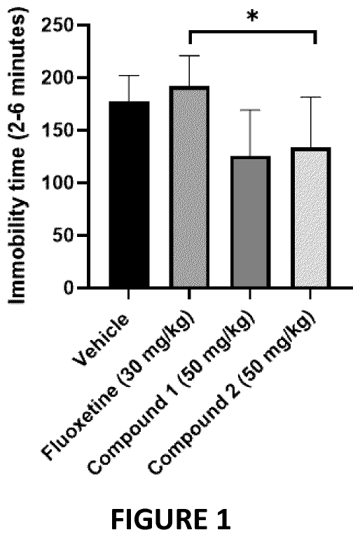

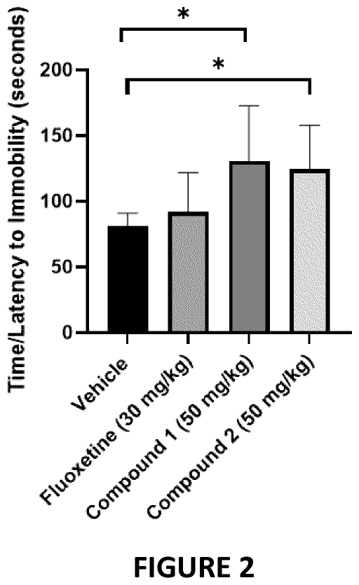

As shown in FIGS. 1 and 2, both Compounds (1) and (2) were significantly better than fluoxetine (Prozac®) on immobilization time (2-6 minutes) and better than vehicle control for latency to immobility. There was no evidence of hallucinatory activity for any compound. The data suggest (1) and (2) are surprisingly fast acting anti-depressants using a one-way ANOVA and Fisher LSD method for pairwise analysis of multiple groups.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. A compound selected from the group comprising Formula (6): 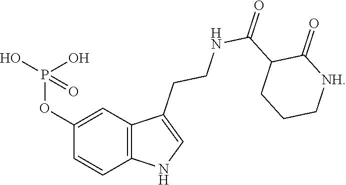 [SMILES: O=C1NCCCC1C(=O)NCCc1c[nH]c2ccc(OP(=O)(O)O)cc12]
Formula (8): 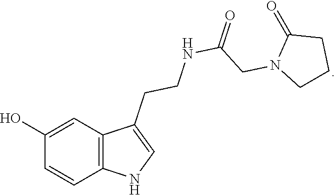 [SMILES: O=C(CN1CCCC1=O)NCCc1c[nH]c2ccc(O)cc12]
and Formula (10):

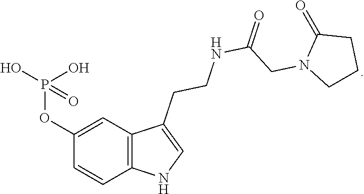 [SMILES: O=C(CN1CCCC1=O)NCCc1c[nH]c2ccc(OP(=O)(O)O)cc12]

2. A method of treating or preventing a disorder or condition comprising administering the compounds of claim 1.

3. Use of the compounds of claim 1 for the treatment or prevention of a disorder or condition.

4. A compound of claim 1 for the treatment or prevention of a disorder or condition.

5. The method, use, or compound of claim 2, wherein the disorder or condition is depression.

6. (canceled)

7. The method, use, or compound of claim 2, wherein the disorder or condition is one of more of Parkinson's disease, amyotrophic lateral sclerosis, Alzheimer's disease, fetal alcohol exposure, autism, schizophrenia, traumatic brain injury, stroke, addictions, Huntington's disease, Fragile X and Rett's syndrome.

8. The method, use, or compound of claim 2, further comprising administration with a compound that modulates neuroplasticity.
